(19) United States
(12) Patent Application Publication
(10) Pub. No.: US 2004/0191254 A1
(43) Pub. Date: Sep. 30, 2004

(54) Method of treatment of thyroid cancer

(76) Inventor: James Alexander Fagin, Cincinnati, OH (US)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/491,859

(22) PCT Filed: Oct. 8, 2002
(86) PCT No.: PCT/US02/32195

Related U.S. Application Data
(60) Provisional application No. 60/327,880, filed on Oct. 9, 2001.

(51) Int. Cl. A61K 39/395; A61K 31/00; C12Q 1/68

(52) U.S. Cl. 424/145.1; 514/1

(57) ABSTRACT

The present invention relates to a method of treating a warm-blooded animal, especially a human, having a disease which is mediated or characterized by mutations in the RET gene, or thyroid cancer, especially thyroid cancer harboring RET mutations, comprising administering to said animal a therapeutically effective amount of a compound which decreases the activity of the epidermal growth factor (EGF), especially a compound as defined herein.

The present invention relates to a method of treating a warm-blooded animal, especially a human, having a disease which is mediated or characterized by mutations in the RET gene or thyroid cancer, especially thyroid cancer harboring RET mutations, comprising administering to said animal a therapeutically effective amount of a compound which decreases the activity of the epidermal growth factor (EGF), especially a compound as defined herein.

The human RET gene, localized on chromosome 10q11.2, encodes a transmembrane receptor of the protein tyrosine kinase family. The gene consists of 21 exons, which are transcribed into at least three mRNA variants. The mature glycosylated protein is 170 kD in size, and contains three major domains: an extracellular domain involved in ligand binding that consists of cadherin-like and cysteine-rich regions; a transmembrane domain; and an intracellular portion containing the tyrosine kinase domain (TK) split by a 27 amino acid insertion.

The RET proto-oncogene is involved in the regulation of growth, survival, differentiation and migration of cells of neural crest origin. Four ligands for RET have been identified: the glial cell line derived neurotrophic factor, neurturin, persephin, and artemin. After ligand binding, RET is induced to dimerize, resulting in activation of the kinase activity of the receptor, autophosphorylation at selected tyrosine residues, and initiation of intracellular signaling through interaction of effectors with specific tyrosine-phosphorylated domains of the receptor. The mutations in the RET gene involved in generation of either medullary thyroid cancer or papillary thyroid cancers code for constitutively active receptors in which one of the key regulatory functions that control its activation has been subverted. In sporadic papillary thyroid carcinomas rearrangements of RET resulting in constitutive activation of its tyrosine kinase function (RET/PTC) have been observed. This oncogenic hit is likely involved in disease causation, as demonstrated by the generation of papillary carcinomas in mice with targeted expression of RET/PTC in the thyroid by means of a thyroglobulin gene promoter.

Approximately 18,000 new cases of thyroid cancer are diagnosed each year in the USA. Of these, about 90% are papillary thyroid carcinomas (PTC) arising from thyroid follicular cells. Medullary thyroid carcinomas (MTC) originate from calcitonin-secreting parafollicular C cells, and represent 5 to 10% of all thyroid cancers. About 25% of medullary thyroid carcinomas are hereditary, either as part of multiple endocrine neoplasia type 2 (MEN2), or of familial medullary thyroid carcinoma (FMTC). Germline mutations of the RET proto-oncogene confer predisposition to all hereditary forms of MTC, through an autosomal dominant mode of transmission.

The tyrosine kinase activity of the receptor for epidermal growth factor (EGF) plays a key role in signal transmission in a large number of mammalian cells, including human cells, especially epithelial cells, cells of the immune system and cells of the central and peripheral nervous system. For example, in various cell types, EGF-induced activation of receptor-associated tyrosine protein kinase is a prerequisite for cell division and hence for the proliferation of the cell population. A number of compounds which decreases the activity of the EGF is known in the art.

Surprisingly, it has now been found that a compound which decreases the activity of the EGF, especially an EGF-R tyrosine kinase inhibitor, can be used as a therapeutic agent for the treatment of a disease which is mediated or characterized by mutations in the RET gene, and, in particular, of thyroid cancer.

Hence, the invention relates to the use of a compound which decreases the activity of the epidermal growth factor (EGF) for the preparation of a medicament for the treatment of thyroid cancer and to a method of treating thyroid cancer, especially thyroid cancer harboring RET mutations resulting in constitutive activation of its tyrosine kinase function, comprising administering to a warm-blooded animal, preferably a human, more preferably a male human, in need thereof a therapeutically effective amount of a compound which decreases the activity of the EGF.

A compound which decreases the activity of the EGF is preferably an EGF-R tyrosine kinase inhibitor as disclosed in WO97/02266 or PCT/EP02/08780, very preferably an EGF-R tyrosine kinase inhibitor selected from PKI166, OSI774, C225 (cetuximab), CI-1033, ABX-EGF, EMD-72000, IRESSA™ and MDX-447, more preferably PKI166, OSI774, C225 and IRESSA™. Most preferably, the EGF-R tyrosine kinase inhibitor employed is PKI166.

In one embodiment, the present invention provides in particular a method of treating pediatric thyroid carcinomas. In another embodiment, the present invention provides a method of treating thyroid cancers caused by exposure to radiation. Furthermore, the present invention provides a method of treating hereditary medullary thyroid carcinomas, especially MEN2 and FMTC.

The term “thyroid cancer” as used herein comprises, but is not restricted to, medullary thyroid cancer and papillary thyroid cancer.

The structure of the active ingredients identified by code nos., generic or trade names may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active ingredients and, based on these references, likewise enabled to manufacture and test the pharmaceutical indications and properties in standard test models, both in vitro and in vivo.

The term “compounds which decrease the activity of the EGF” as used herein are compounds which inhibit the EGF receptor tyrosine kinase, compounds which inhibit the EGF receptor and compounds binding to EGF, and are in particular those compounds generically and specifically disclosed in WO 97/02266 (describing compounds of formula I), PCT/EP02/08780, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and, especially, WO 96/33980; in each case in particular in the compound claims and the final products of the working examples, which are hereby incorporated into the present application by reference to this publications. Comprised are likewise the corresponding stereoisomers as well as the corresponding crystal modifications, e.g. solvates and polymorphs, which are disclosed therein. The compounds used as active ingredients in the present invention can be prepared and administered as described in the cited documents, respectively.

The term “treatment” as used herein comprises the treatment of patients having thyroid carcinomas or being in a pre-stage of said disease which treatment effects the delay of progression of the disease in said patients.

In a broader sense, the present invention relates to a method of treating a disease which is mediated or characterized by mutations in the RET gene comprising administering a therapeutically effective amount of a compound which decreases the activity of the epidermal growth factor (EGF) to a warm-blooded animal in need thereof and to the use of a compound which decreases the activity of the EGF for the preparation of a medicament for the treatment of a disease which is mediated or characterized by mutations in the RET gene.

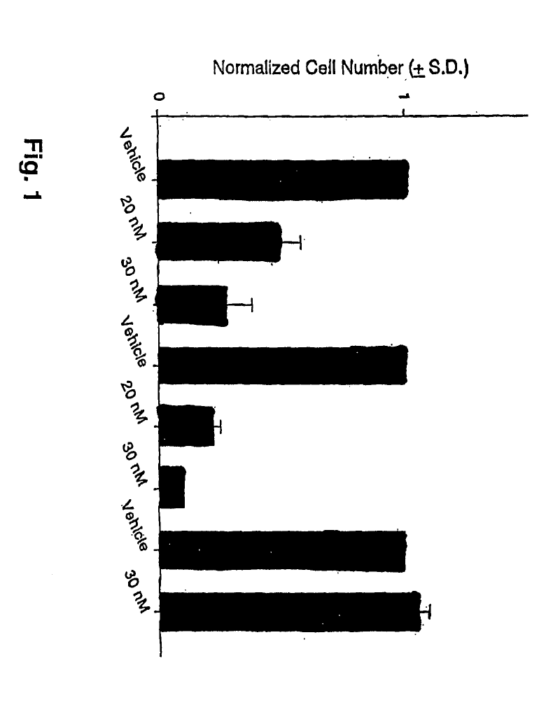

Short description of FIG. 1:

The drawing illustrates the effect of PKI166 on the growth of NIH3T3 cells expressing constitutively active RET Cys634Tyr.

PKI166 inhibits the growth of RET-transformed fibroblasts. The indicated cell lines are allowed to plate overnight in 6-well plates (NIH3T3 cells at 5×10⁴; 3T3-RETC634Y at 2×10⁴). They are then grown in the presence of no PKI166, 20 nM PKI166 or 30 nM PKI166 for 9 days, with media changes every 3 days. Bars represent the X±SD of cell counts in 3 independent experiments. The first three columns show the results in NIH3T3-RetCys634Tyr in 5% serum, the next three columns show the results in NIH3T3-RetCys634Tyr in 1% serum and the last two columns the results in NIH3T3 in 5% serum (only vehicle and 30 nM PKI 166).

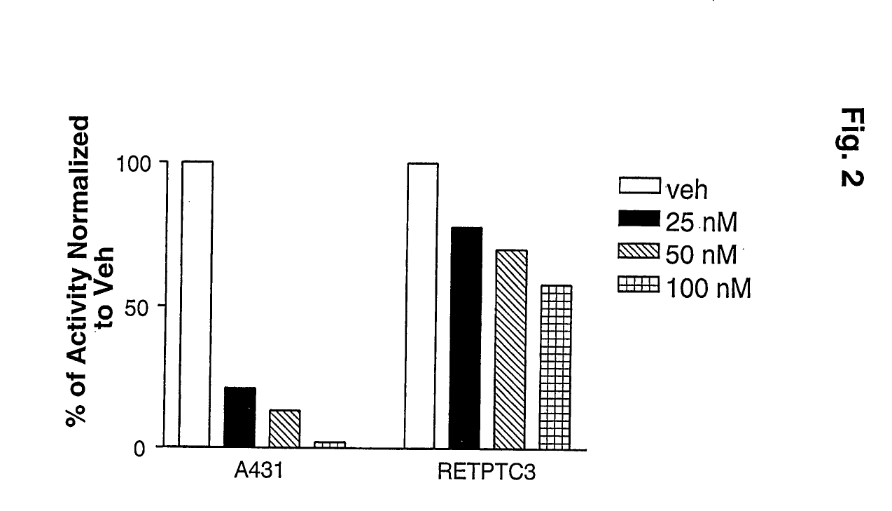

Short description of FIG. 2:

The drawing illustrates the effects of a compound of formula III* on EGF-R and RET kinase activities in A431 and RET PTC3-5 cell line (PCCL3 cells with doxycycline-inducible expression of RET/PTC3).

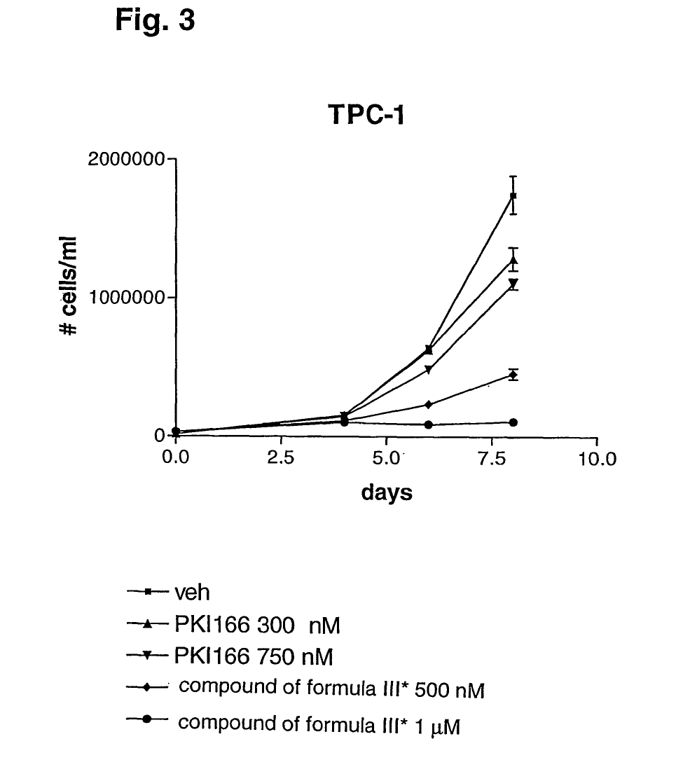

Short description of FIG. 3:

The drawing illustrates the effects of the indicated compounds on growth of PTC-1 cells (papillary thyroid carcinoma cell line with endogenous activation of RET/PTC-1).

A number of peptides are reported to effect the activity of the EGF. Peptides have the disadvantage to get easily hydrolyzed under physiological conditions, especially those physiological conditions to be found in the blood or stomach of warm-blooded animals. Therefore, such compounds are preferred in the present invention which are no peptides.

The potency of the compound to inhibit the EGF tyrosine kinase can, e.g., be evaluated by incubating compounds with the tyrosine kinase in the presence of [³³P]-ATP and an artificial substrate, using optimised buffer and salt conditions. Phosphorylated tyrosine on the substrate is then detected by means of a β-scintillation counter. The drug concentration required to inhibit the EGF enzyme activity by 50% (IC50 value) of compounds which inhibit the EGF receptor tyrosine kinase as defined herein is typically between 10 and 150 nM, preferably between about 15 and 50 nM.

Unless stated otherwise, in the present disclosure organic radicals and compounds designated “lower” contain not more than 7, preferably not more than 4, carbon atoms.

In one embodiment of the invention, compounds which inhibit the EGF receptor tyrosine kinase are in particular 7H-pyrrolo[2,3-d]pyrimidine derivatives of formula I

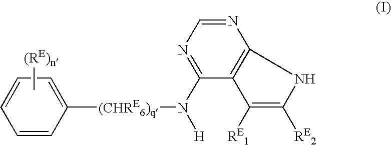

wherein

q′ is 0 or 1,

n′ is from 1 to 3 when q′ is 0, or n′ is from 0 to 3 when q′ is 1,

R^{E} is halogen, lower alkyl, hydroxy, lower alkanoyloxy, lower alkoxy, carboxy, lower alkoxycarbonyl, carbamoyl, N-lower alkyl-carbamoyl, N,N-di-lower alkyl-carbamoyl, cyano, amino, lower alkanoylamino, lower alkylamino, N,N-di-lower alkylamino or tri-fluoromethyl, it being possible when several radicals R^{E} are present in the molecule for those radicals to be identical or different,

a) R^{E}₁ and R^{E}₂ are each independently of the other

α) phenyl substituted by carbamoyl-methoxy, carboxy-methoxy, benzyloxycarbonyl-methoxy, lower alkoxycarbonyl-methoxy, phenyl, amino, lower alkanoylamino, lower alkylamino, N,N-di-lower alkylamino, hydroxy, lower alkanoyloxy, carboxy, lower alkoxycarbonyl, carbamoyl, N-lower alkyl-carbamoyl, N,N-di-lower alkyl-carbamoyl, cyano or by nitro;

β) hydrogen under the proviso that R^{E}₁ and R^{E}₂ cannot represent hydrogen at the same time;

γ) unsubstituted or halo- or lower alkyl-substituted pyridyl;

δ) N-benzyl-pyridinium-2-yl; naphthyl; cyano; carboxy; lower alkoxycarbonyl; carbamoyl; N-lower alkyl-carbamoyl; N,N-di-lower alkyl-carbamoyl; N-benzyl-carbamoyl; formyl; lower alkanoyl; lower alkenyl; lower alkenyloxy; or

ε) lower alkyl substituted by

εα) halogen, amino, lower alkylamino, piperazino, di-lower alkylamino,

εβ) phenylamino that is unsubstituted or substituted in the phenyl moiety by halogen, lower alkyl, hydroxy, lower alkanoyloxy, lower alkoxy, carboxy, lower alkoxycarbonyl, carbamoyl, N-lower alkyl-carbamoyl, N,N-di-lower alkyl-carbamoyl, cyano, amino, lower alkanoylamino, lower alkylamino, N,N-di-lower alkylamino or by trifluoromethyl,

εγ) hydroxy, lower alkoxy, cyano, carboxy, lower alkoxycarbonyl, carbamoyl, N-lower alkyl-carbamoyl, N,N-di-lower alkyl-carbamoyl, mercapto or

εδ) by a radical of the formula R^{E}₃—S(O)_{m′}— wherein R^{E}₃ is lower alkyl and m′ is 0, 1 or 2, or

b) when q′ is 0, one of the radicals R^{E}₁ and R^{E}₂ is unsubstituted lower alkyl or unsubstituted phenyl and the other of the radicals R^{E}₁ and R^{E}₂ has one of the meanings given above in paragraph a) with the exception of hydrogen, or

c) when q′ is 1, R^{E}₁ and R^{E}₂ are each independently of the other unsubstituted phenyl or have one of the meanings given above in paragraph a), and

R^{E}₆ is hydrogen, lower alkyl, lower alkoxycarbonyl, carbamoyl, N-lower alkyl-carbamoyl or N,N-di-lower alkyl-carbamoyl,

and to the salts thereof.

The radicals and symbols as used in the definition of a compound of formula I have the meanings as disclosed in WO 97/02266 which publication is hereby incorporated into the present application by reference.

The term “PKI166” as used herein means a EGF receptor tyrosine inhibitor of formula I wherein q′ is 1, n′ is 0, R^{E}₁ is hydrogen, R^{E}₂ is phenyl substituted by 4-hydroxy, and R^{E}₆ is methyl.

A very preferred EGF receptor tyrosine inhibitor of formula I is PKI166 {(R)-6-(4-hydroxy-phenyl)-4-[(1-phenyl-ethyl)-amino]-7H-pyrrolo[2,3-d]-pyrimidine)}.

A further preferred EGF receptor tyrosine inhibitor of formula I is a compound of formula I, wherein q′ is 1, n′ is 0, R^{E}₁ is hydrogen, R^{E}₂ is phenyl substituted by CH₃—CH₂—CO—NH—, and R^{E}₆ is methyl.

In another embodiment of the invention, compounds which inhibit the EGF receptor tyrosine kinase are in particular quinazoline derivatives of the formula II

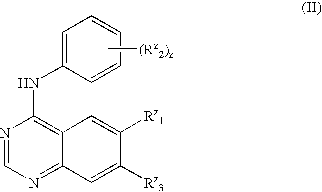

wherein

z is 1, 2 or 3 and each R^{z}₂ is independently halogen, trifluoromethyl or C₁-C₄alkyl;

R^{z}₃ is C₁-C₄alkoxy; and

R^{z}₁ is C₁-C₄alkoxy; di-(C₁-C₄alkyl)amino-C₂-C₄alkoxy, pyrrolidin-1-yl-C₂-C₄alkoxy, piperidino-C₂-C₄alkoxy, morpholino-1-yl-C₂-C₄alkoxy, piperazin-1-yl-C₂-C₄alkoxy, 4-C₁-C₄alkylpiperazin-1-yl-C₂-C₄alkoxy, imidazol-1-yl-C₂-C₄alkoxy, di-[(C₁-C₄alkoxy)-C₂-C₄alkyl]amino-C₂-C₄alkoxy, thiamorpholino-C₂-C₄alkoxy, 1-oxothiamorpholino-C₂-C₄al or 1,1-dioxothiamorpholino-C₂-C₄alkoxy, and wherein any of the above-mentioned R^{z}₁ substituents comprising a methylene group which is not attached to a N or O atom optionally bears on said methylene group a hydroxy substituent,

or a pharmaceutically acceptable salt thereof.

The radicals and symbols as used in the definition of a compound of formula II have the meanings as disclosed in WO 96/33980 which publication is hereby incorporated into the present application by reference.

Preferably, a compound of formula II is employed wherein R^{z}₁ and R^{z}₃ are both methoxy and R^{z}₂ is bromo or a pharmaceutically acceptable salt thereof.

More preferably, a compound of formula II is employed which is 4-(3′-chloro-4′-fluoro-anilino)-7-methoxy-6-(3-morpholinopropoxy)-quinazoline or a pharmaceutically acceptable salt thereof.

In another embodiment of the invention, compounds which inhibit the EGF receptor tyrosine kinase are in particular compounds of the formula III

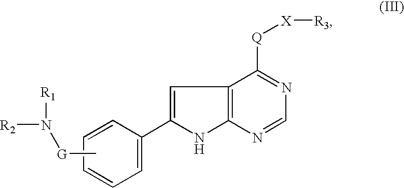

wherein

R₁ and R₂ are each independently of the other hydrogen, unsubstituted or substituted alkyl or cycloalkyl, a heterocyclic radical bonded via a ring carbon atom, or a radical of the formula R₄—Y—(C═Z)— wherein R₄ is unsubstituted, mono- or disubstituted amino or a heterocyclic radical, Y is either not present or lower alkyl and Z is oxygen, sulfur or imino, with the proviso that R₁ and R₂ are not both hydrogen; or

R₁ and R₂ together with the nitrogen atom to which they are attached form a heterocyclic radical;

R₃ is a heterocyclic radical or an unsubstituted or substituted aromatic radical;

G is C₁-C₇-alkylene, —C(═O)—, or C₁-C₆-alkylene-C(═O)— wherein the carbonyl group is attached to the NR₁R₂ moiety;

Q is —NH— or —O—, with the proviso that Q is —O— if G is —C(═O)— or C₁-C₆-alkylene-C(═O)—; and

X is either not present or C₁-C₇-alkylene, with the proviso that a heterocyclic radical R₃ is bonded via a ring carbon atom if X is not present;

or a salt of the said compounds.

The radicals and symbols as used in the definition of a compound of formula III have the meanings as disclosed in EP02/08780 which publication is hereby incorporated into the present application by reference.

Preferably, a compound of formula III is employed wherein R₁ and R₂ together with the nitrogen atom to which they are attached form a 4-lower alkyl-piperazinyl radical, R₃ is phenyl, G is methylene, Q is —NH— and X is —CH(CH₃)—, which, in the present specification, is referred to as “a compound of formula III*”, or a pharmaceutically acceptable salt thereof.

It will be understood that in the discussion of methods, references to the active ingredients are meant to also include the pharmaceutically acceptable salts. If these active ingredients have, for example, at least one basic center, they can form acid addition salts. Corresponding acid addition salts can also be formed having, if desired, an additionally present basic center. The active ingredients having an acid group (for example COOH) can also form salts with bases. The active ingredient or a pharmaceutically acceptable salt thereof may also be used in form of a hydrate or include other solvents used for crystallisation.

The pharmaceutical compositions according to the present invention can be prepared in a manner known per se and are those suitable for enteral, such as oral or rectal, and parenteral administration to warm-blooded animals, including man, comprising a therapeutically effective amount of at least one pharmacologically active ingredient, alone or in combination with one or more pharmaceutically acceptable carries, especially suitable for enteral or parenteral application. The preferred route of administration of the dosage forms of the present invention is orally.

The person skilled in the pertinent art is fully enabled to select relevant test models to prove the beneficial effects mentioned herein on a disease which is mediated or characterized by mutations in the RET gene, e.g. thyroid cancer, of a compound which decreases the activity of the EGF. The pharmacological activity of such a compound may, for example, be demonstrated by means of the Examples described below, by in vivo tests in nude or transgenic mice or in suitable clinical studies. Suitable clinical studies are, for example, open label non-randomized, dose escalation studies in patients with metastatic medullary thyroid carcinoma. The efficacy of the treatment is determined in these studies, e.g., by radiologic evaluation of the tumors every 6 weeks or by suitable serum tumor markers with the control achieved on placebo matching with the active ingredient.

The effective dosage of the compounds which decrease the activity of the EGF may vary depending on the particular compound or pharmaceutical composition employed, e.g., the mode of administration, the type of the thyroid cancer being treated or the severity of the thyroid cancer being treated. The dosage regimen is selected in accordance with a variety of further factors including the renal and hepatic function of the patient. A physician, clinician or veterinarian of ordinary skill can readily determine and prescribe the effective amount of compounds which decrease the activity of the EGF required to prevent, counter or arrest the progress of the condition. Optimal precision in achieving concentration of the active ingredients within the range that yields efficacy without toxicity requires a regimen based on the kinetics of the active ingredients' availability to target sites. The dosage of a compound of formula I is preferably in the range of about 50 to 700, more preferably about 100 to 500, and most preferably about 150 to 300, mg/day. The applied oral dosage of Iressa™ (ZD1839) is preferably the one as described in the package insert for the treatment of tumor diseases.

In order to explore the activity of a compound which decreases the activity of the EGF on RET kinase, for example, a well-differentiated clonal thyroid cell line, PCCL3, conditionally expressing either RET/PTC3 or RET/PTC1 in a tetracyclin (doxycyclin)-dependent manner as described below can be used. The activation of expression of RET/PTC1 or 3 results in dimerization, autophosphorylation, and association with a number of signaling intermediates including Shc and PLCγ.

PCCL3 cell lines are maintained in H4 complete medium consisting of Coon's medium/F12 high zinc supplemented with 5% FBS, 0.3 mg/ml L-glutamine, 1 mlU/ml TSH, 10 μg/ml insulin, 5 μg/ml apo-transferrin, 10 nM hydrocortisone, and penicillin/streptomycin. The expression system used was developed by Bujard and co-workers to deliver doxycyclin-inducible expression based on the high specificity of interactions of the E. coli tet repressor-operator with doxycyclin. Stable transfections are performed first to establish clonal lines constitutively expressing the transactivator rtTA (composed of a fusion of the rtetR DNA binding domain and the VP16 activation domain). Individual rtTA-expressing clones are then explored for doxycyclin-inducible expression by transient transfection with a luciferase reporter construct under control of a tet-operator. Clones of rtTA demonstrating very low or undetectable basal luciferase activity and marked induction (i.e. >100 fold) by doxycyclin are selected as hosts for secondary stable transfection with constructs consisting of a minimal CMV promoter containing tet-operator sequences cloned upstream of either RET/PTC1 or RET/PTC3 cDNAs.

The human squamous-cell carcinoma cell line A431 stably overexpressing the EGF-R is grown in DMEM supplemented with 10% fetal calf serum at 37 C in a 5% CO2 atmosphere. RET/PTC1 and RET/PTC3 oligomerizes and displays constitutive tyrosine kinase activity. The insulin receptor overexpressing cell line CHO-wt IR is grown in Ham's F-12 medium with 10% fetal bovine serum.

### EXAMPLES

### Example 1

### Inhibition of Autophosphorylation of EGFR (A431 cells) or RetPTC3-5 (PCCL3) by EGF-R Tyrosine Kinase Inhibitors

Confluent T-75 flasks are washed with ice cold PBS containing 0.2 mM sodium ortho-vanadate, Cells are then lysed with cold RIPA buffer 1.8 ml (20 mM Tris, pH 7.4, 150 mM NaCl, 1% Nonidet P-40, 1% Tween 20, 20 mM sodium fluoride, 1 mM sodium ortho-vanadate, 1 mM EGTA, 5 mM EGTA, 0.2 mM PMSF, with Sigma Protease inhibitor mix) with constant agitation at 4 C for 20 min. Cell lysates are passed through a 26-gauge needle to disperse large aggregates, and centrifuged for 30 minutes at 10,600×G, 4 C. The cleared supernatants are incubated with anti-RET antibody (Santa Cruz goat polyclonal) or anti-EGFR (Santa Cruz) for 2 h at 4 C and then incubated with proteinAG agarose (Santa Cruz) previously washed with RIPA buffer. The immuno-complexes are spun, washed twice in washing buffer (50 mM HEPES, pH 7.2, 20 mM MnCl₂, 5 mM MgCl₂) and once with kinase buffer (washing buffer plus 0.5 mM dithiothreitol). Immunocomplexes pelleted after the final wash are resuspended in kinase buffer and aliquotted to reaction tubes. Kinase assays are performed in a 20 μl incubation buffer containing 0.5% DMSO with or without the indicated concentration of the inhibitor. Reactions are performed in duplicate by the addition P³²-ATP (Perkin-Elmer; >6000 Ci/mmol) with a specific activity of 140 nCi/pmol for 25 minutes at room temperature. Reactions are stopped by with two washes with STOP Buffer (10 mM phosphate buffer pH7, 1% TritonX-100, 0.1% sodium deoxycholate, 1 mM sodium orthovanate, 1 mM ATP, 5 mM EDTA, and 5 μg/ml aprotinin). After the second wash, proteins are eluted by boiling in 35 μl Laemmli buffer for 10 minutes. Proteins are subjected to SDS-PAGE gel (7.5%), their phosphorylation measured by Phosphorimager densitometry (Molecular Dynamics, Sunnyvale, Calif.) after transfer to nitrocellulose membranes. Phosphorylation is then normalized to total RET protein in the IP determined by Western analysis using goat polyclonal anti-RET antibody (SantaCruz).

The effects of PKI166 on RET/PTC autophosphorylation are examined in such in vitro immunokinase assays of RET-IP extracts from RET/PTC3-5 cells treated with doxycycline for 48 h to maximally induce expression of the oncoprotein. No kinase activity in RET-IP lysates is observed in untreated cells. IC50 of CPG75166 on RET/PTC3 is approximately 17.7 nM. By contrast, IC50 of the compound on EGF-R autophosphorylation in immunokinase assays of A431 cells is 8 nM. PKI66 has no effects on insulin receptor autophosphorylation in immunokinase assays of CHO-wt-IR cells.

### Example 2

### Effects of EGF-R Tyrosine Kinase Inhibitors on Activation of PLCγ by RET/PTC

Ret-PTC3-5 cells are seeded at 1×10⁵ cells/well in 6-well Corning plates. After 3 days, cells are treated with or without doxycycline in the presence of the selected concentration of inhibitor dissolved in solvent for 24 h. Cells are rinsed twice with cold PBS containing 0.1 mM sodium orthovanadate, and left for 20 minutes in ice-cold RIPA buffer. Cell lysates are collected by centrifugation at 4C, and pelletted at 10,000×g for 20 min. Protein assays are performed on aliquots of supernatants by the Coomassie Blue assay (Pierce, Rockford, Ill.). 650 μg of protein are incubated with anti-PLC γ antibody (SantaCruz) or normal IgG overnight. The immune complexes are precipitated with proteinAG agarose (Santa Cruz) previously washed with RIPA buffer as described. After three washes with RIPA buffer, precipitates are eluted into 30 μl sample buffer, heated 10 min at 95 C, and ran on SDS-PAGE gel for Western blot analysis. Blots are initially probed with anti-phosphotyrosine. Loading is normalized by probing with anti-PLC γ antibody (SantaCruz).

It was shown before that upon activation, RET associates with and phosphorylates PLCγ. To further explore the effects of PKI166 on RET kinase activity, the impact of pretreatment with the compound on PLCγ phosphorylation is examined. When grown in the absence of doxycycline, there is no detectable PLCγ phosphorylation. Pretreatment with PKI166 inhibits PLCγ phosphorylation in a dose-dependent fashion, with an IC50 of approximately 4 nM.

### Example 3

### Effects of PKI166 on Growth of NIH3T3-RETC634L Cells

RETC634L is the most common germine mutation of RET in multiple endocrine neoplasia type 2A stably expressing a constitutively active form of RET. NIH3T3-RETC634Y cells are transformed, as evidenced by growth in low serum conditions, colony formation in soft agar, and tumor formation in nude mice. Treatment of these cells with PKI166 evokes a powerful, concentration dependent inhibition of cell growth. PKI166 has no effect on growth of wild-type NIH3T3 cells grown in 5% serum (FIG. 1).

1. Use of a compound which decreases the activity of the epidermal growth factor (EGF) for the preparation of a medicament for the treatment of thyroid cancer.

2. The use according to claim 1 wherein the thyroid cancer harbors RET mutations.

3. The use according to claim 1 wherein the thyroid cancer is hereditary medullary thyroid cancer.

4. The use according to claim 1 wherein the thyroid cancer is caused by exposure to radiation.

5. Use of a compound which decreases the activity of the epidermal growth factor (EGF) for the preparation of a medicament for the treatment of a disease which is mediated or characterized by mutations in the RET gene.

6. The use according to claim 1 wherein the compound which decreases the activity of the EGF is an EGF-R tyrosine kinase inhibitor selected from PKI166, OSI774, C225, CI-1033, ABX-EGF, EMD-72000, IRESSA™ and MDX-447.

7. A method of treating thyroid cancer comprising administering a therapeutically effective amount of a compound which decreases the activity of the epidermal growth factor (EGF) to a warm-blooded animal in need thereof.

8. The method according to claim 8 wherein the thyroid cancer harbors RET mutations.

9. The method according to claim 8 or 9 wherein the thyroid cancer is hereditary medullary thyroid cancer.

10. The method according to claim 7 wherein the thyroid cancer is caused by exposure to radiation.

11. A method of treating a disease which is mediated or characterized by mutations in the RET gene comprising administering a therapeutically effective amount of a compound which decreases the activity of the epidermal growth anyone of-factor (EGF) to a warm-blooded animal in need thereof.

12. The method according to claim 7 wherein the warm-blooded animal is a human.

13. The method according to claim 12 wherein the human is younger than 18 years.

14. The method according to claim 7 wherein the compound which decreases the activity of the EGF is an EGF-R tyrosine kinase inhibitor selected from PKI166, OSI774, C225, CI-1033, ABX-EGF, EMD-72000, IRESSA™ and MDX-447.
